(19) United States
(12) Patent Application Publication
(10) Pub. No.: US 2014/0288582 A1
(43) Pub. Date: Sep. 25, 2014

(54) Implant Localization Device

(71) Applicants: Michael T. Milbocker, Holliston, MA (US); Lukas von Bluecher, Eurasburg (DE)

(72) Inventors: Michael T. Milbocker, Holliston, MA (US); Lukas von Bluecher, Eurasburg (DE)

(73) Assignee: BVW HOLDING AG, Cham (CH)

(21) Appl. No.: 14/129,041

(22) PCT Filed: Jun. 21, 2012
(86) PCT No.: PCT/US12/43471

§ 371 Date: Jun. 6, 2014

Related U.S. Application Data
(60) Provisional application No. 61/499,648, filed on Jun. 21, 2011.

(51) Int. Cl. A61F 2/00; A61L 31/14

(52) U.S. Cl. CPC A61F 2/0063; A61L 31/148
USPC 606/151

(57) ABSTRACT

An implantable medical device for providing structural support to tissue is described wherein the implant adheres to tissue without the benefit of suture, surgical adhesive and the like. The adherent or localization means is a hemostat or protein polymerizing materials such as oxidized cellulose, alpha cellulose, polyanhydroglucuronic acid and the like. The structure of the hemostat may be fibular (hallow and solid), woven, particulate, and the structure of hemostats in general. These protein polymerization compounds are beneficially attach to at least one side of a planar implant such as a surgical repair mesh, surgical barrier, and any implantable device intended to isolate or strength a layer of mammalian tissue.

### CROSS-REFERENCE TO RELATED APPLICATIONS

This application claims the benefit of U.S. Provisional Application No. 61/499,648 (Att. Docket MB8564PR), filed on Jun. 21, 2011 and entitled Implant Localization Device, and is related to U.S. Provisional Application No. 61/496,435 (Att. Docket MB8560PR), filed on Jun. 13, 2011 and entitled POLYOL MODIFIED NATURAL BOSWELLIC ACID COMPLEXES, the entire contents of both which are hereby incorporated by reference.

### BACKGROUND OF THE INVENTION

1. Field of the Invention

One of the primary problems with tissue separating and reinforcing implantable devices is their localization to a surgical site. Typically, suture, stables, tissue adhesives and the like are used to fix these devices to a location within the body. These currently used methods of localizing an implant in the body are associated with adverse clinical outcomes. Sutures and staples localize stresses commonly applied to such devices which can lead to mobilization of the implant resulting in adhesions, failure of the tissue repair, and generally re-operation and pain. Tissue adhesives are expensive and difficult to use. Ideally the localization means is a functionality of the implant without the need for auxiliary localization means.

The present invention is preferably an implantable medical device comprised of biodegradable materials and a hemostat for strengthening or isolating a layer of tissue in a mammalian body, Hemostats polymerize aqueous proteins which is a useful feature for adhering an implant to a tissue site. Oxidized cellulose, alpha cellulose, polyanhydroglucuronic acid and the like are commercially available hemostats.

Solid hemostats can be imbedded in most absorbable materials, either during casting in a solvated state or during extrusion in a melted state. The resulting implant device should be stable under common storage conditions, have a predictable controlled degradation profile, provide localization of the implant to a tissue plane within minutes, and allow the planar implant to be rolled, delivered through a trocar and easily unrolled within the body, be easily moved to a tissue site without excessive adherence, and be quickly localized to the site once the implant is placed. Surgeons are well practiced in the use of solid hemostats, and in particular hemostats of sheet-like geometry. The handling characteristics of these hemostats are suitable for a diverse range of applications in the surgical treatment of tissue within a body. Surgeons are well practiced in the use of absorbable surgical barriers and soft tissue reinforcement devices. The present invention is a novel use of a hemostat utilizing its polymerization activity to localize an implantable planar device within a body, and not utilizing necessarily its hemostatic activity, wherein the two are mechanically or chemically joined.

2. Description of Related Art

Biocompatible, biodegradable polymers have been widely used in the medical field as surgical barriers, soft tissue repair mesh, protective membranes for the treatment of wounds, and drug delivery systems. Among biodegradable polymers, polylactide, polyglycolide and a copolymer of lactide and glycolide, are all commercially available. They have good biocompatibility and are decomposable in the body to harmless materials such as carbon dioxide, and water. Hemostats comprised of oxidized cellulose, alpha cellulose and polyanhydroglucuronic acid are also absorbable and biocompatible.

The following are issued patents and applications related to the present invention.
U.S. Pat. No. 5,660,854 and 6,534,693 describe a surgical implant or external wound dressing which functions as both a hemostat and a device to safely and effectively deliver any of a number of pharmaceuticals to targeted tissue at a controlled rate is disclosed. The device generally comprises an implant in the form of fibers, sutures, fabrics, cross-linked solid foams or bandages, a pharmaceutical in solid micoparticulate form releasably bound to the implant fibers, and a lipid adjuvant which aids the binding of the microparticles to the fibers as well as their function in the body.
U.S. Pat. No. 5,795,286 is a radioisotope impregnated material sheet or mesh designed to be placed between internal body tissues to prevent the formation of post-operative adhesions. This mesh or gauze into which the isotope is placed may be either a permanent implant or it may be biodegradable. One embodiment is realized by impregnating an existing product such as the Johnson & Johnson SURGICEL™ absorbable hemostat gauze-like sheet with a beta emitting radioisotope such as phosphorous-32,
U.S. Pat. No. 5,972,366 describes a surgical implant or external wound dressing which functions as both a hemostat and a device to safely and effectively deliver any of a number of pharmaceuticals to targeted tissue at a controlled rate is disclosed.
U.S. Pat. No. 4,093,576 describes a doughy bone cement mixture formed by mixing a powder-form polymer, such as polymethyltrimethacrylate, with a polymerizing liquid monomer, such as a liquid monomeric methylmethacrylate, to form a water-insoluble composition and admixing this composition with oxidized cellulose.
U.S. Pat. No. 4,882,167 describes a hydrophobic carbohydrate polymer, e.g. ethyl cellulose; and, generally at least one digestive-difficulty soluble component, i.e., a wax, e.g. carnauba wax, fatty acid material or neutral lipid provides upon dry direct compression a controlled and continuous release matrix for tablets or implants of biologically active agents,
U.S. Pat. No. 5,282,857 describes a medical implant, which comprises an outer envelope and a gel filler material, wherein the gel comprises water and a cellulose gelling agent.
U.S. Pat. No. 5,380,328 describes a composite surgical implant structure for use in orthognathic and reconstructive surgery, the implant structure is comprised of at least one layer of perforated, biocompatible metallic sheet material and at least one layer of biologically and chemically inert microporous membrane material in intimate contact with, and supported by, the layer of perforated metallic sheet material. The microporous membrane material is comprised of randomly dispersed polytetrafluoroethylene fibers, or mixtures of cellulose acetate and cellulose nitrate fibers,
U.S. Pat. No. 5,658,329 describes a soft tissue implant filling material. The material may be polyvinylpyrollidone, polyvinyl alcohol, hydroxypropylmethyl cellulose, polyethylene oxide, hyaluronic acid, sodium or calcium alginate, hydrogel polyurethane, hydroxyethyl starch, polyglycolic acid, polyacrylamide, hydroxyethylmethacrylate (HEMA), and several naturally derived biopolymers including sodium kinate, seaweed, and agar.
U.S. Pat. No. 5,766,631 describes a wound implant materials comprising a plurality of bioabsorbable microspheres bound together by a bioabsorbable matrix, such as in a freeze-dried. collagen matrix. The microspheres and/or the matrix preferably comprise a polylactic/polyglycolic copolymer, collagen, cross-linked collagen, hyaluronic acid, cross-linked hyaluronic acid, an alginate or a cellulose derivative.

In consideration of the related art, it is an object of this invention to have a sheet of material that can be placed between internal body tissues, the material having a hemostat attached to localize the device between adjacent layers of human tissue.

Another object of this invention is to have a biodegradable sheet of material or mesh suitable for placement between body tissues including an attached hemostat wherein the attached hemostat is oxidized cellulose, alpha cellulose or polyanhydroglucuronic acid.

### SUMMARY OF THE INVENTION

It has been recognized that it would be advantageous to develop an implantable soft tissue repair or surgical barrier with a localization means comprising a hemostat capable of polymerizing aqueous proteins when administered into a particular body site.

A first embodiment of this invention is a device consisting of a hemostat impregnated into, coated onto or placed onto a material sheet or mesh designed to be placed between internal body tissues that have been surgically separated to prevent the post-operative mobility of the implant. A hemostat that is impregnated into a smooth sheet of material or coated onto the material or joined to the material by adhesion, bonding and/or absorption is defined herein as a hemostat “attached” to a surgical barrier.

This sheet onto which the hemostat is attached may be either a permanent implant or it may be preferably biodegradable. The hemostat can be attached to an existing product such as the MAST Biosurgery SurgiWrap™ absorbable surgical barrier. Hemostats possess many beneficial attributes, including package stability, insensitivity to sterilization methods, and ability to adhere to a tissue site by absorption and polymerization. Implant mobility has been associated with tissue adhesions between a tissue repair and surrounding tissue. It is beneficial to combine a medical implant such as a surgical barrier with hemostats and their derivatives.

The sheet onto which the hemostat is attached can be comprised of biodegradable polymeric compositions which may include organic esters or ethers, which when degraded result in physiologically acceptable degradation products, including the monomers. Anhydrides, amides, orthoesters or the like, by themselves or in combination with other monomers, may find use. The polymers are alternatively condensation polymers. The polymers may be cross-linked or non-cross-linked, usually not more than lightly cross-linked, generally less than 15%, usually less than 5%.

Still another embodiment of this invention is to attach a hemostat composition to a device such as a soft tissue reconstructive mesh that is used for the treatment of a hernia. Since scar tissue formation is one of the main complications of hernia repair, by attaching a hemostat composition to a. mesh that is placed over a tissue defect, the use of suture or staples may be eliminated and some reduction in adhesion severity and incidence realized.

The polylactic acid polyurethane copolymer in the present invention is preferably a well distributed random block copolymer of a hydrophilic poly(alkylene glycol) blocks and a hydrophobic polylactic acid blocks dispersed such that the polymeric ends are randomly hydrophilic and hydrophobic. Preferably these polymers are formed into a sheet to which is attached a fibrous hemostat, such as oxidized cellulose.

The content of the hemostat is preferably within the range of 0.1 to 50% by weight and more preferably from 1 to 10% by weight, based on the total weight of the composition. The molecular weight of the biodegradable polymer is within the range of 500 to 5,000,000 Daltons and is preferably from 1,000 to 50,000 Daltons.

The implants may be monolithic, i.e. having the hemostat homogenously distributed through the polymeric matrix, or applied to one or both side of the polymeric surface, where the hemostat is bonded, or impregnated during formation of the implant.

Additional features and advantages of the invention will be apparent from the detailed description which follows.

### DETAILED DESCRIPTION

Reference will now be made to the exemplary embodiments, and specific language will be used herein to describe the same. It will nevertheless be understood that no limitation of the scope of the invention is thereby intended. Further, these examples do not limit alterations and further modifications of the inventive features illustrated herein, and additional applications of the principles of the inventions as illustrated herein, which would occur to one skilled in the relevant art and having possession of this disclosure, are to be considered within the scope of the invention.

The present invention makes use of biodegradable materials that will be gradually dissolved in the body of a living subject, and which can be impregnated with a hemostat composition, thereby resulting in localization of an implantable sheet formed from biodegradable materials. Suitable biodegradable materials will gradually disassociate in vivo, and will not have any substantial toxic or other harmful effect on the subject. Examples of suitable biodegradable materials are polylactic acid, polyglycolic acid, dilactic acid, and lactic acid-glycolic acid copolymers. Polyglycolic acids having molecular weights between 1000 and 50,000 Daltons are preferred. Dilactic acid/polyglycolic acid ratios of 75/25 and 85/15 by weight are commercially available and are useful in the present invention. Additional suitable materials are those with good mechanical properties which have been modified to breakdown in the body. For instance, copolymers of those materials mentioned previously and polyurethanes, or polyurethanes synthesized with diisocyanate with a degradable link between the isocyanate groups.

The hemostat of the present invention is preferably insoluble in melts and solutions of the polymer, if the polymer component is prepared as a solution during formation of the implant. Preferably the polymer portion is also water-insoluble. The polymer matrix should be stable during storage, during sterilization, and should not degrade in the body significantly over a period of at least 2 days, preferably at least 2 weeks for instance a month or more.

In one particular embodiment, fibers of oxidized cellulose are pressed into a sheet of polylactic acid using heat, such that the oxidized cellulose melts partially into the surface of the polylactic acid sheet. The application of the oxidized cellulose is such that the fibers partially protrude from the polylactic acid sheet, thereby providing a certain roughness to the otherwise smooth sheet which aids in localizing the sheet. Furthermore, exposure of the oxidized fibers serves to polymerize proteins dissolved in the fluids attaching the implant to tissue.

In another exemplary embodiment, a woven oxidized cellulose fabric is pressed into a sheet of polylactic acid using heat, such that one side of the oxidized cellulose fabric melts partially into the surface of the polylactic acid sheet. The application of the oxidized cellulose fabric is such that the woven structure partially protrudes from the polylactic acid sheet, thereby providing a certain roughness to the otherwise smooth sheet which aids in localizing the sheet. Furthermore, exposure of the oxidized fibers serves to polymerize proteins dissolved in the fluids attaching the implant to tissue. Additionally, the oxidized cellulose fabric may be melted into the polymeric sheet only at discrete locations, for example, at raised portions of the oxidized cellulose fabric. When portions of the oxidized cellulose are not adhered to the polymeric sheet, interstitial spaces are created around which tissue may grow into and around, thereby further localizing the implant over a longer period.

In another exemplary embodiment, the oxidized cellulose is applied along the margin of an implant, for example, on a 1 cm widestripe on the perimeter of the implant.

In another exemplary embodiment, the oxidized cellulose is applied as discretepads, such as circles, adhered in a regular pattern over the surface of the polymeric implant.

In another aspect, additional embodiments are directed to polymeric implants having a hydrophilic coating applied to a side of the absorbable hydrophobic polymer sheet wherein oxidized cellulose is imbedded. The operational principle relies on the hydrophilic layer acting as an attractant to draw aqueous proteins into one side of the implant where the oxidized cellulose serves to polymerize proteins contained in the aqueous fluid to form a solid bridge between implant and tissue.

In another aspect, the hydrophilic layer may be a hydrogel in a dessicated state whereby when the hydrogel becomes hydrated in situ it becomes adhesive and conformable to an irregular tissue surface, It may be further conformable by swelling so as to fill a tissue defect, thereby bringing the imbedded oxidized cellulose in close proximity to a tissue surface which may not generally be in contact over the entire surface of a substantially planar implant.

In general, hemostats are hydrophobic, and can be made less so by addition of polyether chains, in particular polyethylene oxide. Variations in hydrophilicity can be achieved by grafting onto the hemostats polyether chains comprising varying ratios of polyethylene oxide and polypropylene oxide. The greater the proportion of propylene oxide to ethylene oxide in a copolymeric polyether is, the more hydrophobic the final composition of polyether chain and hemostat. In this way, the modified oxidized cellulose may serve both as the protein polymerizing component and the hydrogel component.

In another aspect, the oxidized cellulose may be imbedded in a layer of collagen, or like biologic material, which is known to aid in the healing of a defect in tissue. Polymer sheets modified with a biologic may aid in healing, and in some instances promote angiogenesis, which is a critical aspect of healthy, stable tissue remodeling. It has been recognized that regenerated tissue devoid of cells is inherently unstable and undergoes a continuous process of remodeling, which is associated with pain. An implant that promotes angiogenesis, and hence blood flow, will result in repair tissue which is rich in cells and far less likely to remodel.

In particular, a material such as SiS extracellular matrix (Cook Biotech, West Lafayette, Ind.) is a highly porous multilayer biologic implant used in soft tissue repair. Polylactic acid dissolved in a suitable solvent could be applied between a layer of polylactic acid such as SurgiWrap™ (MAST Biosurgery, San Diego, Calif.) and SiS extracellular matrix such that the polylactic acid solution dissolves partially into the SurgiWrap and absorbs partially into the SiS extracellular matrix thereby bonding the two together when the solvent is driven off. To the opposite side of the SiS extracellular matrix is applied a sheet of oxidized cellulose. Attachment can be achieved by pressing into the SiS extracellular matrix, sewn in place, or bonded using the mentioned method using a solution of a polymer or other biocompatible adhesive. The resulting device possesses a surgical barrier to adhesions on one side, and tissue scaffold sandwiched between a layer of oxidized cellulose and the polylactic acid sheet. The oxidized cellulose provides localization of the composite structure.

Other polymers useful in conjunction with a polymeric implant sheet include polymeric coatings such as ethylene vinyl acetate copolymers, copolymers of ethylene and alkyl acrylate or polyalkylmethacrylate, copolymers of ethylene and propylene, styrene butadiene rubber, or silicone based polymers.

Organic solvents useful in the manufacture of the above described embodiments include, but not limited to, halogenated hydrocarbons, aromatic and aliphatic hydrocarbons, alcohols, cyclic ethers, ketones, such as methylene chloride, ethanol, tetrahydrofuran, toluene, acetone and 1,1,2 trichloroethane.

Modification of the hydrophobicity of the polymer implant can be accomplished by adding conditioning polymer, Useful conditioning polymers include biostable polymers which are also biocompatible such as, but not limited to, polyurethanes, silicones, ethylene-vinyl acetate copolymer, polyethers such as homopolymers or copolymers of alkylene oxide, homo- or copolymers of acrylic, polyamides, polyolefins, polyesters, polydienes, cellulose and related polymers.

Bioabsorbable polymers that could be used include poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-cotrimethytene carbonate), polyphosphoester, polyphosphoester urethane, poly(amino acids), cyanoacrylates, poly(trimethylene carbonate), poly(iminocarbonate), copoly(ether-esters), polyalkylene oxalates, polyphosphazenes and biomolecules such as fibrin, fibrinogen, cellulose, starch, collagen and hyaluronic acid. These and other polymer systems can be used if they can be dissolved or dispersed in a solvent system hosting the primary polymeric implant.

Alternatives to using a polylactic acid sheet are absorbable polyurethanes. Absorbable polyurethanes can be synthesized by grafting a single glycolide, lactide or caprolactone between two isocyanate groups. More particularly, single isocyanate groups are attached to aromatic or aliphatic rings and these mono-isocyanates are bridged by glycolide, lactide, caprolactone or low molecular weight co- or ter-polymers of these. Then polyurethane can be synthesized by reacting polyethers with these degradable diisocyanates without the presence of monomers. The polyethers can be copolymers of ethylene oxide and propylene oxide without the presence of monomeric contaminants.

The materials of the present invention have thermal properties that allow processing of the material in melt form at relatively low temperatures, or in solvent systems thus avoiding trans-esterification and other side-reactions that cause the generation of undesired degradation and other by-products. At the same time, the thermal properties are such that the materials can be used to imbed a hemostat such as oxidized cellulose.

### EXAMPLES

The use of ahemostatto localized a planar implant is illustrated in the Examples that follow. In these Examples, in some instances only a small amount of the hemostat is imbedded on a side of the planar implant. In other Examples, the hemostat is used in combination with longer term implant localizing architectures, such as a tissue scaffold. By the Examples provided it is shown the present invention can be adapted to a variety of implant compositions suited to diverse surgical applications.

While the following preparations and examples are provided for the purpose of illustrating certain aspects of the present invention, they are not to be construed as limiting the scope of the appended claims. The chemical used in these examples can be obtained from Sigma-Aldrich, Milwaukee, Wis., unless otherwise stated.

### EXAMPLE 1

### Preparation of an Adhering Solution

50 g of D,L-lactide recrystallized from ethyl acetate was added to 50 g of acetone and dissolved to make a clear, colorless, viscous solution.

### EXAMPLE 2

### Attachment of Oxidized Cellulose to a Surgical Barrier

A sheet of polylactic acid surgical barrier (SurgiWrap, MAST Biosurgery, San Diego, Calif.) is placed on a glass sheet. A sheet of oxidized cellulose polymer (Surgical, Ethicon, Cincinnati, Ohio) is painted on one side with the solution of Example 1 and the resulting coated. oxidized cellulose applied to the sheet of polylactic acid under 5 psi pressure. The pressure is maintained until the solvent leaves the construct, thereby bonding the oxidized cellulose to the polylactic acid sheet.

### EXAMPLE 3

### Attachment of Oxidized Cellulose to a Surgical Barrier

A sheet of polylactic acid surgical barrier (SurgiWrap, MAST Biosurgery, San Diego, Calif.) is placed on a glass sheet. A sheet of oxidized cellulose polymer (Surgical, Ethicon, Cincinnati, Ohio) is on top of the polylactic acid surgical barrier. Under 5 psi pressure heat is applied to the oxidized cellulose side until the oxidized cellulose melts partially into the polylactic acid sheet, thereby bonding the oxidized cellulose to the polylactic acid.

### EXAMPLE 4

### Attachment of Oxidized Cellulose to a Composite Surgical Barrier

A sheet of polylactic acid surgical barrier (SurgiWrap, MAST Biosurgery, San Diego, Calif.) is placed on a glass sheet. A sheet of SIS extracellular matrix (Cook Biotech, West Lafayette, Ind.) is painted on one side with the solution of Example 1 and resulting coated SiS extracellular matrix is applied to one side of the sheet of polylactic acid under 5 psi pressure. The pressure is maintained until the solvent leaves the construct, thereby creating a composite surgical barrier consisting of an anti-adhesion layer of polylactic acid on one side and a tissue scaffold of SiS extracellular matrix on the other side.

A sheet of oxidized cellulose polymer (Surgicell, Ethicon, Cincinnati, Ohio) is painted on one side with the solution of Example 1 and the resulting coated oxidized cellulose applied to the sheet composite surgical barrier on the side where the SiS extracellular matrix resides under 5 psi pressure. The pressure is maintained until the solvent leaves the construct, thereby bonding the oxidized cellulose to the composite surgical barrier.

### EXAMPLE 5

### Absorbable Polyurethane Prepolymer

20 g of ethylene diol (100 MW) and 200 g of low molecular weight D,L-lactide (MW=2000) recrystallized from ethyl acetate were added to a sealed glass reactor equipped with externally driven stir rod, heating jacket and internal thermocouple. The headspace was flushed with dry argon continuously through an oil trap, Thereto was added 0.5 g of stannous octoate (SnOct.sub.2) dissolved in 10 ml of toluene. The reactor was heated in steps to 120 degrees C. with constant stirring at 100 revolutions per minute, Under reduced pressure (15 mmHg), the reaction was continue for 8 hours. The resulting product was dissolved in chloroform. The solution was slowly added to cold acetone (0 degrees C.) to precipitate the formed polymer. The polymer can be further purified by repeating the dissolution-precipitation_{—} process and then drying in vacuo (0.1 mmHg) for 24 hours. The molecular weight of the copolymer (PEG-PLA) was identified by GPC.

The 110 g PEG-PLA polymer synthesized above was dissolved in 100 ml toluene. This solution and 346 g toluene diisocyanate acetate were added to a sealed glass reactor equipped with externally driven stir rod, heating jacket and internal thermocouple. The headspace was flushed with dry argon continuously through an oil trap. The reactor was heated in steps to 60 degrees C. with constant stirring at 100 revolutions per minute. The reaction was continued until the % NCO of the reaction reached 3.3%.

The result is a degradable diisocyanate. This can be used to make a polyurethane copolymer of propylene glycol and ethylene glycol. As a simple example, a degradable polyurethane will be constructed using a pluronic comprised of 25% propylene oxide and 75% ethylene oxide.

196 g of UCON 75-H-450 (MW=980), 381 g of the PEG-PLA diisocyanate synthesized. above and 200 ml toluene were added to a sealed glass reactor equipped with externally driven stir rod, heating jacket and internal thermocouple. The headspace was flushed with dry argon continuously through an oil trap. The reactor was heated in steps to 60 degrees C. with constant stirring at 100 revolutions per minute. The reaction was continued until the % NCO was measured to be below 0.1%. The result is a toluene solution of prepolymer of degradable polyurethane.

### EXAMPLE 6

### Oxidized Cellulose Attached to Absorbable Polyurethane

The solution of Example 5 is placed in a petri dish and let to stand at ambient condition. The toluene is allowed to evaporate until the resulting liquid has a viscosity of approximately 50,000 cps, Then a sheet of oxidized cellulose polymer (Surgicell, Ethicon, Cincinnati, Ohio) is lightly placed on the surface and the oxidized cellulose allowed to wick down to the surface of the polyurethane prepolymer. At ambient conditions, water vapor in the air cures the polyurethane prepolymer and the result is an absorbable polyurethane with oxidized cellulose bonded to one side.

What is claimed is:

1. A tissue isolating or strengthening implantable medical device comprising a planar geometry on at least one side of which is attached a protein polymerizing compound.

2. The medical device of claim 1 wherein the said protein polymerizing compound is a hemostat.

3. The medical device of claim 2 wherein said hemostat is at east one of oxidized cellulose, alpha cellulose, polyanhydroglucuronic acid.

4. The medical device of claim 1 wherein said planar device is a hernia mesh.

5. The medical device of claim 1 wherein said planar device is a surgical barrier.

6. The medical device of claim 1 wherein said protein polymerizing compound is placed on one side.

7. A planar medical device containing a protein polymerizing compound on the surface of one side wherein when said planar medical device is placed onto the surface of a tissue layer, the protein polymerizing compound bonds to tissue thus localizing implant relative to said tissue layer.

8. The medical device of claim 7 wherein said protein polymerizing compound is a fibrous hemostat bonded to one side of said implant.

9. The medical device of claim 8 wherein said fibrous hemostat comprises a solid fiber.

10. The medical device of claim 8 wherein said fibrous hemostat comprises a hallow fiber.

11. The medical device of claim 7 wherein said protein polymerizing compound is woven and bonded to one side of said implant.

12. A planar implantable medical device comprising a polylactic acid sheet and fibular oxidized cellulose wherein said fibular oxidized cellulose is partially imbedded into said polylactic acid sheet such that when said implantable medical device is placed on tissue with the side on which said oxidized cellulose is deposited against said tissue layer said implantable medical device polymerizes to said layer of tissue.

13. A planar implantable medical device comprising a composite surgical barrier comprising on one side an anti-adhesion surface and on the other side a tissue scaffold, said composite surgical barrier bonded to fibular oxidized cellulose wherein said fibular oxidized cellulose is partially imbedded into said composite surgical barrier such that when said implantable medical device is placed on tissue with the side on which said oxidized cellulose is deposited against said tissue layer said implantable medical device polymerizes to said layer of tissue.
